(19) United States
(12) Patent Application Publication
(10) Pub. No.: US 2018/0116916 A1
(43) Pub. Date: May 3, 2018

(54) LEAVE-ON TYPE AEROSOL COSMETIC COMPOSITION

(71) Applicant: COSMAX, INC., Hwaseong-si (KR)

(72) Inventors: Byoung Mo SHIN, Gunpo-si (KR); Youn Joon KIM, Seoul (KR); Hyung Jun KIM, Seongnam-si (KR); Youn Seok NAM, Seoul (KR)

(21) Appl. No.: 15/567,798

(22) PCT Filed: Feb. 18, 2016
(86) PCT No.: PCT/KR2016/001648

§ 371 Date: Oct. 19, 2017

(30) Foreign Application Priority Data
Apr. 22, 2015 (KR) 10-2015-0056554

(51) Int. Cl. A61K 8/04; A61K 8/19; A61K 8/41; A61K 8/44; A61K 8/34; A61K 8/36; A61K 8/92; A61K 8/891; A61K 8/86; A61K 8/60; A61K 8/73; A61K 8/33; A61K 8/31; A61Q 19/00

(52) U.S. Cl. CPC A61K 8/046; A61Q 19/00; A61K 8/41; A61K 8/44; A61K 8/345; A61K 8/361; A61K 8/922; A61K 8/92; A61K 8/891; A61K 8/86; A61K 8/60; A61K 8/73; A61K 8/33; A61K 8/315; A61K 8/19

(57) ABSTRACT

Provided is an aerosol cosmetic composition including carbon dioxide as a propellant and containing at least one ingredient selected from vegetable oil, animal oil and a fatty acid, or an aerosol cosmetic composition further including an alkaline ingredient. The aerosol cosmetic composition causes little skin irritation and can be formulated into a leave-on type formulation which allows direct absorption of a cosmetic product into the skin, and emits little odor unique to the content upon spraying and can provide a glossy foam shape by virtue of the incorporation of carbon dioxide as a propellant.

### TECHNICAL FIELD

The present invention relates to a leave-on type aerosol cosmetic composition. More particularly, the present invention relates to an aerosol cosmetic composition including carbon dioxide as a propellant and containing at least one ingredient selected from vegetable oil, animal oil and a fatty acid, or to an aerosol cosmetic composition further including an alkaline ingredient.

### BACKGROUND ART

Fundamental cosmetics are those imparting various efficacies/effects, including basic effects of moisturizing and glossing, to the skin. Particularly, various formulations, including liquid, lotion, cream and gel, such as skin, lotion, essence and cream, impart efficacies/effects to the skin.

Currently, many studies have been conducted about gas-filled aerosol type cosmetics as rare items under the situation of requiring various types of cosmetics. Most aerosol cosmetics are limited in hair care products and generally provided as hair moose, hair spray, or the like. However, recently, the other formulations, such as moose-like cleanser or pack have been developed actively. In addition, in the case of the existing aerosol product, a gas having a low-boiling point of 0-50° C. is used generally. For example, propane, butane, isobutane or dimethyl ether is used and imparts an excellent effect of forming foam. However, the above-mentioned gases have their unique odors and are somewhat problematic. More recently, there is a trend toward avoiding the use of LPG and dimethyl ether. Thus, it is required to develop aerosol cosmetics using a propellant which has no color and no color and is safe to the skin.

In this context, Korean Patent Publication No. 10-0953144 discloses a foam-type massage moose pack cosmetic composition using carbon dioxide as a propellant. The cosmetic composition contains a large amount of surfactant to and has a formulation not intended to be absorbed into the skin but intended to be washed out.

Therefore, the present inventors have conducted many studies to overcome the above-mentioned problems according to the related art. As a result, we have found that an aerosol cosmetic composition including carbon dioxide as a propellant and containing at least one ingredient selected from vegetable oil, animal oil or a fatty acid, or an aerosol cosmetic composition further including an alkaline ingredient can be formulated into a leave-on type formulation which causes little skin irritation and allows direct absorption of a cosmetic product into the skin, and emits little odor unique to the content upon spraying and can provide a glossy foam shape by virtue of the incorporation of carbon dioxide as a propellant. The present invention is based on this finding.

### REFERENCES

(Patent Document 0001) KR 10-0953144B

### DISCLOSURE

### Technical Problem

A technical problem to be solved by the present invention is to provide a leave-on type aerosol cosmetic composition, which causes little skin irritation and can be formulated into a leave-on type formulation that allows a cosmetic product to be absorbed directly into the skin, contains carbon dioxide as a propellant to emit little odor unique to the content upon spraying, and can provide a glossy foam-like phase.

### Technical Solution

In one general aspect, there is provided an aerosol cosmetic composition including carbon dioxide as a propellant and containing at least one ingredient selected from vegetable oil, animal oil and a fatty acid. There is also provided an aerosol cosmetic composition further including an alkaline ingredient in addition to the above-mentioned aerosol cosmetic composition.

The conventional aerosol type cosmetic product is limited in a hair care product and cleansing product, and the gaseous ingredients and cosmetic ingredients contained in such products emit a unique odor and cause skin irritation, and thus such products are limited in application to cosmetic formulations to be absorbed into the skin. Under these circumstances, according to the present invention, a leave-on type aerosol cosmetic composition sprayed as foam is provided by using carbon dioxide as a propellant and limiting the type and amount of a surfactant.

As used herein, the term ‘leave-on type’ refers to a cosmetic product which is applied to the skin and then is not washed out but is allowed to stay so as to be absorbed into the skin.

According to an embodiment, carbon dioxide is used in an amount of 0.1-10 wt % based on the total weight, the vegetable oil, animal oil or fatty acid is used in an amount of 0.1-10.0 wt % based on the total weight, and the alkaline ingredient is used in an amount of 0.1-5.0 wt % based on the total weight.

When carbon dioxide is used in an amount less than 0.1 wt %, it is not possible to spray the composition in a foam shape. When carbon dioxide is to used in an amount larger than 10 wt %, the composition is not sprayed in a foam shape but is atomized like spray. In addition, it is difficult to fill carbon dioxide to 10 wt % due to the pressure of carbon dioxide. When the vegetable oil, animal oil or fatty acid is used in an amount less than 0.1 wt %, it is not possible to form foam. When the vegetable oil, animal oil or fatty acid is used in an amount larger than 20.0 wt %, foam is formed but a surfactant is generated in an excessive amount undesirably to cause an irritating feeling, which is not suitable for use in a leave-on type formulation. When the alkaline ingredient is used in an amount less than 0.1 wt %, it is not possible to form foam. When the alkaline ingredient is used in an amount larger than 5.0 wt %, an irritating feeling is increased, which is not suitable for use in a leave-on type formulation.

According to another embodiment, any propellant used generally in the conventional aerosol cosmetics may be used, besides carbon dioxide as a propellant. Preferably, LPG, dimethyl ether or tetrafluoropropene may be further incorporated.

According to the test example described hereinafter, in the case of a composition including carbon dioxide as a propellant, it is possible to obtain dense and glossy foam. When carbon dioxide is used in combination with the other propellant, it is possible to obtain a formulation to be realized according to the present invention. As can be seen from such results, not only carbon dioxide alone used as a propellant but also the other propellants used in combination with carbon dioxide can provide dense and glossy foam (see Test Example 1).

According to another embodiment, the vegetable oil, animal oil and fatty to acid may be used alone or in combination.

The aerosol cosmetic composition according to the present invention may include vegetable oil, animal oil or a fatty acid individually or in combination.

According to still another embodiment, the fatty acid is represented by the following Chemical Formula 1 and may be a carboxylic acid obtained by hydrolysis from animal- or plant-derived oil and fat or oil. Preferably, the fatty acid may be lauric acid, myristic acid, palmitic acid, stearic acid, behenic acid, isostearic acid or oleic acid.

The fatty acid has a structure of R—COOH (Chemical Formula 1), wherein R is a saturated or unsaturated, linear or isomeric alkyl having 8-24 carbon atoms. As the length of alkyl is increased (the number of carbon atoms is increased), the structure becomes bulkier and thus the degree of skin irritation is decreased. In general, any surfactant produced from a fatty acid with an alkaline ingredient may be used as a fatty acid.

According to still another embodiment, the vegetable oil may be any conventionally used vegetable oil, and preferred examples thereof include coconut oil, almond oil, apricot seed oil, jojoba oil, macadamia nut oil, meadow foam seed oil, peanut oil, camellia oil, babassu oil, cacao seed butter or corn germ oil. In addition, any conventionally used animal oil may be used and preferred examples thereof include Sesamum indicum oil, mink oil, ostrich oil, emu oil, lanolin, bees wax, badger oil and bear oil. Further, hydrogenated vegetable oil and animal oil may be used.

Since most of the ingredients forming vegetable oil or animal oil are fatty to acids, all of the fatty acids representing them may be included in vegetable oil. In addition, in the case of a butter type, it is similar to vegetable oil or animal oil. This is because vegetable oil and vegetable butter are present in various oil, paste or solid phases depending on the composition and content of unsaturated fatty acids. In the case of the above-mentioned fatty acids, vegetable oil or animal oil, they have an anionic surfactant-like structure when they are neutralized with an alkaline ingredient. Such a structure is a foam-forming ingredient and can provide a shape of foam when it is filled to aerosol, mixed with gases and sprayed.

According to still another embodiment, the alkaline ingredient may be any alkaline ingredient used conventionally for neutralization, and preferred examples thereof include potassium hydroxide, triethanol amine, aminomethyl propanol, tromethamine, sodium hydroxide, aminomethyl propanediol, aminoethyl propanediol, aminopropanediol, bis-hydroxyethyl tromethamine, triisopropanolamine, arginine, tetrahydroxypropyl ethylene diamine, PEG-15 cocamine, diisopropanol amine and ammonia. More preferably, the alkaline ingredient includes at least one sleected from the group consisting of potassium hydroxide, triethanol amine, aminomethyl propanol, aminoethyl propanediol, aminopropanediol, triisopropanol amine, PEG-15 cocamine, tromethamine, arginine and sodium hydroxide.

According to still another embodiment, the composition is sprayed in the form of moose (foam).

According to the Examples described hereinafter, it can be seen that not only the cosmetic composition including carbon dioxide as a propellant and to containing at least one ingredient selected from vegetable oil, animal oil and a fatty acid but also the cosmetic composition further including an alkaline ingredient in addition to the above-mentioned composition form a foam-like phase. As can be seen from the result, it is possible to obtain a foam-like phase to be realized according to the present invention not only from an aerosol cosmetic composition containing an alkaline ingredient but also from an aerosol cosmetic composition free from an alkaline ingredient (see Examples 1-8).

According to still another embodiment, the composition is a leave-on type composition.

According to the Examples described hereinafter, it is shown that Examples including, as a surfactant, vegetable oil, animal oil or a fatty acid, or Examples including, as surfactants, vegetable oil, animal oil and a fatty acid cause low irritation in an irritating feeling test. However, Comparative Example 4 including a large amount of fatty acid and Comparative Examples 5-8 including a surfactant other than a fatty acid or vegetable oil cause skin irritation. It can be seen from the foregoing that the cosmetic composition according to the present invention is suitable for a leave-on type composition which allows a cosmetic product to be absorbed into the skin.

As described earlier, the cosmetic composition according to the present invention may be obtained from a combination with a surfactant capable of forming foam. However, when the cosmetic composition is limited to a leave-on type formulation which is not washed out but is allowed to be absorbed into the skin, there is a limitation in selection of the ingredients. This is because most of anionic surfactants cause skin irritation and may generate skin troubles to accompanied with erythema and urtication upon exposure for a long time, and thus are not amenable to application to a non-wash type product.

In the case of the cosmetic composition according to the present invention, it is formulated into aerosol but should have basic functions as a fundamental cosmetic product. Thus, the cosmetic composition may further include various materials, such as a moisturizing ingredient and nourishing ingredient, in addition to the above-described surfactants. In addition, the composition according to the present invention may optionally include conventional ingredients known to those skilled in the art for maintaining fundamental physical properties and quality, including ingredients used conventionally for preparing a skin cleanser, such as ethylene diamine tetraacetate (EDTA), moisturizer, fat material, wetting agent, silicone, oil, fragrance, preservative, clarifying agent (sugar, alcohol), vitamin, protein, pH modifier or pigment. In addition to the above-mentioned ingredients, the composition according to the present invention may include an anti-oxidative ingredient, skin conditioning ingredient, skin protecting ingredient, UV protecting ingredient, or the like.

### Advantageous Effects

According to the embodiments of the present invention, it is possible to provide a leave-on type formulation which causes little skin irritation and allows a cosmetic product to be absorbed directly into the skin, and to obtain glossy foam emitting little odor unique to the content upon spraying.

### DESCRIPTION OF DRAWINGS

FIG. 1 shows the results of Test Example 1 according to the present invention.

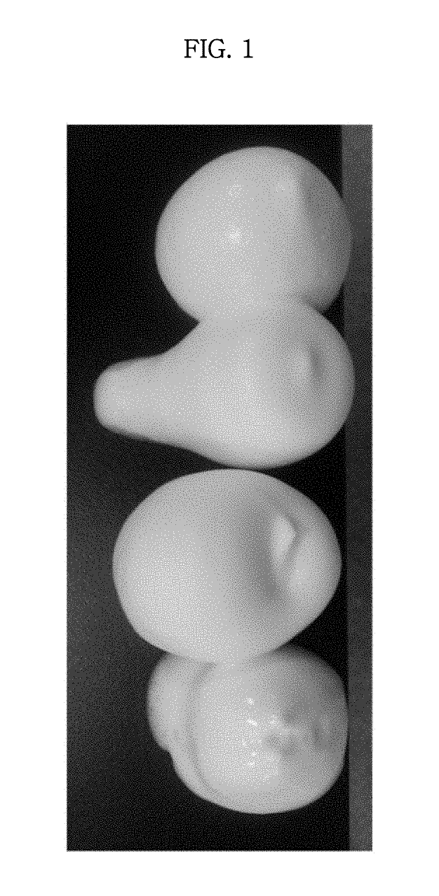

### MODES FOR INVENTION

Hereinafter, the present invention will be explained in more detail with reference to Examples. The following Examples are for illustrative purposes only and not intended to limit the scope of the present invention.

### EXAMPLES 1-4

### Preparation of Foam-Type Cosmetic Compositions

Cosmetic compositions were prepared from the ingredients as shown in the following Table 1.

Triethanolamine (alkali) and glycerine (moisturizing agent) were introduced to purified water and warmed to 80° C. Then, at least one ingredient selected from fatty acids (stearic acid, lauric acid, palmitic acid, oleic acid, isostearic acid and behenic acid), vegetable oil (coconut oil) and animal oil (Sesamum indicum), dimethicone (silicone), PEG-100 stearate/glycerin stearate and Polysorbate 60 (emulsifier) were introduced thereto and the resultant mixture was agitated for about 10 minutes. After agitation, the mixture was cooled, a fragrance was introduced at 40° C. and the resultant mixture was cooled to 30° C. and agitated to provide content. The content was introduced to a pressure-resistant can container, valve was clapped thereto and a propellant was filled thereto to complete a cosmetic agent.

TABLE 1

| No | Ingredients | Ex. 1 | Ex. 2 | Ex. 3 | Ex. 4 | Ex. 5 | Ex. 6 | Ex. 7 | Ex. 8 |
|---|---|---|---|---|---|---|---|---|---|
| 1 | Purified water | To 100 | To 100 | To 100 | To 100 | To 100 | To 100 | To 100 | To 100 |
| 2 | Triethanolamine | 1.0 | 1.2 | 1.0 | 0.5 | 1.0 | 1.5 | 1.3 | — |
| 3 | Potassium | — | — | — | 0.2 | — | — | — | — |
| | hydroxide | | | | | | | | |
| 4 | Arginine | — | — | — | — | 2.0 | — | — | — |
| 5 | Aminomethyl | — | 0.3 | — | — | — | — | — | — |
| | propanol | | | | | | | | |
| 6 | Glycerin | 10.0 | 10.0 | 10.0 | 10.0 | 10.0 | 10.0 | 10.0 | 10.0 |
| 7 | Stearic acid | 3.0 | — | 3.0 | — | 3.0 | — | — | 3.0 |
| 8 | Lauric acid | — | — | — | 1.0 | — | 1.0 | — | — |
| 9 | Palmitic acid | — | — | — | 2.5 | — | — | — | — |
| 10 | Oleic acid | — | — | — | — | 0.5 | 1.0 | — | — |
| 11 | Isostearic | — | — | — | — | 0.5 | — | — | — |
| | acid | | | | | | | | |
| 12 | Behenic acid | — | — | — | — | 0.5 | 1.0 | — | — |
| 13 | Coconut oil | — | 3.0 | 2.0 | — | — | 2.0 | — | — |
| 14 | Sesamum | — | — | — | — | — | — | 3.0 | — |
| | indicum oil | | | | | | | | |
| 15 | Mineral oil | 10.0 | 10.0 | 10.0 | 10.0 | 10.0 | 10.0 | 10.0 | 5.0 |
| 16 | Dimethicone | 1.0 | 1.0 | 1.0 | 1.0 | 1.0 | 1.0 | 1.0 | 5.0 |
| 17 | Fragrance | qsad | qsad | qsad | qsad | qsad | qsad | qsad | qsad |
| 18 | PEG-100 | 0.3 | 0.3 | 0.3 | 0.3 | 0.3 | 0.3 | 0.3 | 0.3 |
| | stearate/glyceryl | | | | | | | | |
| | stearate | | | | | | | | |
| 19 | Polysorbate | 1.0 | 1.0 | 1.0 | 1.0 | 1.0 | 1.0 | 1.0 | 1.0 |
| | 60 | | | | | | | | |
| 20 | Carbon | 2.5 | 2.5 | 2.5 | 2.5 | 2.5 | 2.5 | 2.5 | 2.0 |
| | dioxide | | | | | | | | |
| 21 | LPG | — | — | 5.0 | — | — | — | — | — |
| 22 | Dimethyl | — | — | — | — | 5.0 | — | — | — |
| | ether | | | | | | | | |
| 23 | Tetrafluoro- | — | 5.0 | — | — | — | — | — | — |
| | propene | | | | | | | | |

Example 1 includes a fatty acid as a surfactant in combination with carbon dioxide as a propellant, Example 2 includes a fatty acid and vegetable oil as surfactants in combination with carbon dioxide and tetrafluoropropene as propellants, Example 3 includes a fatty acid and vegetable oil as surfactants in combination with carbon dioxide and LPG as propellants, Example 4 includes a fatty acid as a surfactant in combination with carbon dioxide as a propellant, Example 5 includes a fatty acid as a surfactant in combination with carbon dioxide and dimethyl ether as propellants, Example 6 includes a fatty acid and to vegetable oil as surfactants in combination with carbon dioxide as a propellant, Example 7 includes Sesamum indicum oil as animal oil and carbon dioxide as a propellant, and Example 8 includes a fatty acid as a surfactant and carbon dioxide as a propellant in the absence of an alkaline ingredient.

### COMPARATIVE EXAMPLES 1-8

### Preparation of Foam-Type Cosmetic Compositions

Triethanolamine (alkali) and glycerine (moisturizing agent) were introduced to purified water and warmed to 80° C. Then, materials, such as stearic acid, lauryl phosphate, sodium laureth sulfate, cocamidopropyl betaine and lauryl glucoside (surfactant), were introduced thereto and agitation was carried out for 10 minutes. Then, mineral oil and dimethicone were introduced and the resultant mixture was agitated for 10 minutes and cooled to provide to content. The content was introduced to a pressure-resistant can container, valve was clapped thereto and a propellant was filled thereto to complete a cosmetic agent.

TABLE 2

| | | Comparative Examples | | | | | | | |
|---|---|---|---|---|---|---|---|---|---|
| | | 1 | 2 | 3 | 4 | 5 | 6 | 7 | 8 |
| 1 | Purified water | To 100 | To 100 | To 100 | To 100 | To 100 | To 100 | To 100 | To 100 |
| 2 | Triethanolamine | 1.0 | 1.0 | 1.0 | 5.5 | 1.0 | — | — | 2.0 |
| 3 | Glycerine | 10.0 | 10.0 | 10.0 | 10.0 | 10.0 | 10.0 | 10.0 | 10.0 |
| 4 | Stearic acid | 3.0 | 3.0 | 3.0 | 12.0 | 3.0 | — | — | — |
| 5 | Lauryl phosphate | — | — | — | — | — | — | — | 3.0 |
| 6 | Sodium laureth | — | — | — | — | — | 5.0 | 5.0 | — |
| | sulfate | | | | | | | | |
| 7 | Cocamidopropyl | — | — | — | — | 5.0 | 5.0 | 5.0 | — |
| | betaine | | | | | | | | |
| 8 | Lauryl glucoside | — | — | — | — | 5.0 | — | — | — |
| 9 | Mineral oil | 10.0 | 10.0 | 10.0 | 10.0 | 10.0 | 10.0 | 10.0 | 10.0 |
| 10 | Dimethicone | 1.0 | 1.0 | 1.0 | 1.0 | 1.0 | 1.0 | 1.0 | 1.0 |
| 11 | PEG-100 | 0.3 | 0.3 | 0.3 | 0.3 | 0.3 | 0.3 | 0.3 | 0.3 |
| | stearate/glyceryl | | | | | | | | |
| | stearate | | | | | | | | |
| 12 | Polysorbate 60 | 1.0 | 1.0 | 1.0 | 1.0 | 1.0 | 1.0 | 1.0 | 1.0 |
| 13 | Carbon dioxide | — | — | 11.0 | 2.5 | 2.5 | 2.5 | 2.5 | 2.5 |
| 14 | LPG | — | 5.0 | — | — | — | — | 2.0 | — |
| 15 | Dimethyl ether | — | — | — | — | 5.0 | — | — | — |
| 16 | Tetrafluoropropene | 5.0 | — | — | — | — | — | — | — |

Comparative Examples 1 and 2 include no carbon dioxide and use another propellant, Comparative Example 3 includes carbon dioxide in an amount departing from an adequate range, Comparative Example 4 includes a fatty acid in an amount departing from an adequate range, and Comparative Examples 5-8 include an irritating surfactant (lauryl phosphate).

Information about the ingredients used herein is provided below.

1. Triethanolamine was TEA available from BASF Co.

2. Stearic acid was available from Acid Chem. Co.

3. Lauryl phosphate was Miphos ML available from Miwon Commercial Co., Ltd.

4. Sodium laureth sulfate was Micolin ES528 available from Miwon Commercial Co., Ltd.

5. Cocamidopropyl betaine was Mitain CA available from Miwon to Commercial Co., Ltd.

6. Lauryl glucoside was Plantacare 1200UP available from BASF Co.

7. PEG-100 stearate/glyceryl stearate was Arlacel 165 available from CRODA Co.

8. Polysorbate 60 was Tween 60 available from CRODA Co.

### TEST EXAMPLE 1

### Determination of Foam Shape

The content obtained from each of Examples 1-8 and Comparative Examples 1-8 was introduced to a pressure-resistant can container, valve was clapped thereto and a propellant was filled thereto.

After filling, the content was sprayed and checked whether it was sprayed in the form of foam (moose). The results are shown in the following Tables 3 and 4.

TABLE 3

| | Ex. 1 | Ex. 2 | Ex. 3 | Ex. 4 | Ex. 5 | Ex. 6 | Ex. 7 | Ex. 8 |
|---|---|---|---|---|---|---|---|---|
| Foam | Glossy | Glossy | Glossy | Glossy | Glossy | Glossy | Glossy | Glossy |
| formation | and | and | and | and | and | and | and | and |
| and state | dense | dense | dense | dense | dense | dense | dense | dense |
| | foam | foam | foam | foam | foam | foam | foam | foam |
| | was | was | was | was | was | was | was | was |
| | formed | formed | formed | formed | formed | formed | formed | formed |

TABLE 4

| | Comp. | Comp. | Comp. | Comp. | Comp. | Comp. | Comp. | Comp. |
|---|---|---|---|---|---|---|---|---|
| | Ex. 1 | Ex. 2 | Ex. 3 | Ex. 4 | Ex. 5 | Ex. 6 | Ex. 7 | Ex. 8 |
| Foam | Non- | Non- | No | Glossy | Glossy | Glossy | Glossy | Glossy |
| formation | glossy | glossy | foam | and | but non- | but non- | but non- | but non- |
| and state | foam | foam | was | dense | dense | dense | dense | dense |
| | was | was | formed | foam | foam | foam | foam | foam |
| | formed | formed | | was | was | was | was | was |
| | | | | formed | formed | formed | formed | formed |

As shown in the above Tables, the compositions of Table 1 and Table 2 provided foam upon spraying, and Comparative Examples 5-8 including carbon dioxide provided glossy foam but had a difficulty in absorption as a leave-on type formulation due to an irritating feeling and whitening. In addition, Comparative Examples 1 and 2 allowed use as a leave-on type formulation while forming foam, but it was not possible to obtain glossy foam on the surface, unlike Examples including carbon dioxide. On the contrary, Example 1 using carbon dioxide alone and Examples 2, 3 and 4 using carbon dioxide in combination with another propellant provided glossy foam in the same manner. In the case of Comparative Examples 3 and 4 using an excessive amount of carbon dioxide or a fatty acid. Comparative Example 3 using an excessive amount of carbon dioxide had a difficulty in filling due to a high internal pressure of the container and showed a content-flying phenomenon caused by the strong internal pressure, while not forming foam, when it was filled into a larger container. Thus, it is difficult to obtain foam to be realized according to the present invention. In the case of Comparative Example 4 using an excessive amount of fatty acid, it provided gloss and dense foam but had a difficulty in use to as a leave-on type formulation due to the production of a large amount of soap. Rather, Comparative Example 4 is like a cleansing product departing from the scope of the present invention.

Further, in the case of Examples, it was possible to obtain dense and glossy foam. When each Example was used for massaging and absorbed to the skin, it showed a soft massaging feeling without whitening and did not cause pruritis and reddish skin. However, in the case of Comparative Examples, it was possible to obtain foam-type content upon spraying but it was not possible to obtain dense and glossy content. Particularly, there was a significant difference between a formulation including carbon dioxide and a formulation including no carbon dioxide. Foam formed by an anionic surfactant capable of bubbling was rich but caused whitening upon massaging and absorption, and thus had a difficulty in commercialization as a leave-on type formulation. Particularly, such foam was problematic in that it caused skin irritation including pruritis after several minutes.

As can be seen from the Test Example for Examples and Comparative Examples, some combinations are cable of forming foam but there are limited combinations capable of providing glossy and dense foam. When approaching absorbable products, not wash-out products, the ingredients satisfying desired functions are more limited.

### TEST EXAMPLE 2

### Irritation Test

A skin patch test was carried out for comparison of irritation feelings. The test method is as follows. A patch was attached to a forearm for 24 hours. to Then, 30 minutes, 24 hours and 48 hours after removing the patch, the forearm was checked according to the terminology defined in the ICDRG guideline and evaluated according to the following criteria (Table 5). The evaluated score of response was calculated according to the following Formula 1 to determine the mean score of response for each content. The Results are shown in the following Table 7.

TABLE 5

| Symbol(grade) | Clinical observation |
|---|---|
| −(0) | None |
| ±(0.5) | Doubtful reaction, faint erythema only |
| +(1) | Weak (non-vesicular) reaction, erythema, |
| | slight infiltration |
| ++(2) | Extreme (bullous or ulcerative) |
| +++(3) | Intense erythema with edema & vesicles |

$$Mean score = \frac{\sum ( Score \times No . of Responders )}{\begin{matrix}3 ( Maximum grade ) \times n ( No . of {Total}_{jects} ) \times \\ 3 ( No . of Evalulation )\end{matrix}} \times 100 \qquad [ Formula 1 ]$$

The mean scores of response at 30 minutes, 24 hours and 48 hours were calculated by using the above Formula and the mean score of response for each sample was evaluated according to the criteria of Table 6.

TABLE 6

| Range of Mean Score | Criteria |
|---|---|
| 0.00 ≤ R < 1.01 | Low irritation |
| 1.01 ≤ R < 2.01 | Light irritation |
| 2.01 ≤ R < 3.51 | Medium irritation |
| 3.51 ≤ R | Strong irritation |

TABLE 7

| | | | | | | | | | Comp. | Comp. | Comp. | Comp. | Comp. | Comp. | Comp. | Comp. |
|---|---|---|---|---|---|---|---|---|---|---|---|---|---|---|---|---|
| | Ex. 1 | Ex. 2 | Ex. 3 | Ex. 4 | Ex. 5 | Ex. 6 | Ex. 7 | Ex. 8 | Ex. 1 | Ex. 2 | Ex. 3 | Ex. 4 | Ex. 5 | Ex. 6 | Ex. 7 | Ex. 8 |
| Decision | Low | Low | Low | Low | Low | Low | Low | Low | Low | Low | Low | Medium | Medium | Medium | Medium | Medium |
| | irri- | irri- | irri- | irri- | irri- | irri- | irri- | irri- | irri- | irri- | irri- | irri- | irri- | irri- | irri- | irri- |
| | tation | tation | tation | tation | tation | tation | tation | tation | tation | tation | tation | tation | tation | tation | tation | tation |

As can be seen from Table 7, the aerosol cosmetic composition including, as a surfactant, a fatty acid or vegetable oil, or a fatty acid in combination with vegetable oil is shown to be low irritating and causes little irritation upon skin application, while Comparative Example 4 including an excessive amount of fatty acid and Comparative Examples 4-8 including another type of surfactant cause medium irritation upon skin application.

1. A leave-on type aerosol cosmetic composition comprising carbon dioxide as a propellant and containing at least one ingredient selected from vegetable oil, animal oil and a fatty acid.

2. The leave-on type aerosol cosmetic composition according to claim 1, which further comprises an alkaline ingredient.

3. The leave-on type aerosol cosmetic composition according to claim 2, wherein carbon dioxide is used in an amount of 0.1-10 wt % based on the total weight, the vegetable oil, animal oil or fatty acids used in an amount of 0.1-20.0 wt % based on the total weight, and the alkaline ingredient is used in an amount of 0.1-5.0 wt % based on the total weight.

4. The leave-on type aerosol cosmetic composition according to claim 1, which further comprises LPG, dimethyl ether or tetrafluoropropene in addition to carbon dioxide, as a propellant.

5. The leave-on type aerosol cosmetic composition according to claim 1, which comprises the vegetable oil, animal oil or fatty acid alone or in combination.

6. The leave-on type aerosol cosmetic composition according to claim 1, wherein the fatty acid is represented by the following Chemical Formula 1 and is a carboxylic acid obtained by hydrolysis from animal- or plant-derived oil and fat or oil: 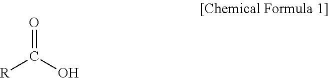
wherein R is a saturated or unsaturated, linear or isomeric alkyl having 8-24 carbon atoms.

7. The leave-on type aerosol cosmetic composition according to claim 2, wherein the alkaline ingredient comprises at least one selected from the group consisting of potassium hydroxide, triethanol amine, aminomethyl propanol, aminoethyl propandiol, aminopropanediol, triisopropanolamine, PEG-15 cocamine, tromethamine, arginine and sodium hydroxide.

8. The leave-on type aerosol cosmetic composition according to claim 1, which is sprayed in the form of moose (foam).

9. (canceled)
